(19) United States
(12) Patent Application Publication
(10) Pub. No.: US 2024/0091406 A1
(43) Pub. Date: Mar. 21, 2024

(54) MULTI-COMPONENT HYDROGEL GEL AND USES OF THE SAME

(71) Applicant: Galderma Holding SA, Zug (CH)

(72) Inventors: Katarina Hågestam, Uppsala (SE); Paula Knutson, Uppsala (SE); Björn Lundgren, Uppsala (SE); Peter Morgan, Uppsala (SE)

(21) Appl. No.: 18/371,340

(22) Filed: Sep. 21, 2023

Related U.S. Application Data
(60) Provisional application No. 63/408,718, filed on Sep. 21, 2022.

(51) Int. Cl. A61L 27/26; A61L 27/52

(52) U.S. Cl. CPC A61L 27/26; A61L 27/52; A61L 2300/236; A61L 2400/06; A61L 2430/34

(57) ABSTRACT

The present disclosure relates to multicomponent hydrogels that may be use, for example, as dermal fillers for treating fine lines and wrinkles of the skin. Also disclosed herein are methods for making and using the disclosed hydrogels and related compositions.

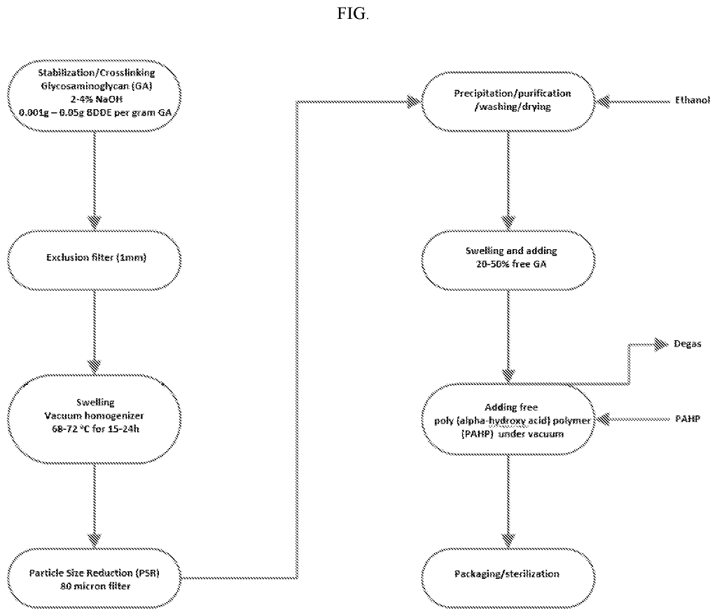

### CROSS-REFERENCE TO RELATED APPLICATIONS

This application claims the benefit of U.S. Provisional Patent Application No. 63/408,718, filed on Sep. 21, 2022, which is hereby incorporated by reference in its entirety.

### TECHNICAL FIELD

The present disclosure relates to hydrogels and compositions comprising the same. The disclosed hydrogels and compositions may be uses as dermal fillers or in cosmetic surgeries, and they can be effective for treating fine lines and wrinkles on the skin.

### BACKGROUND

The following discussion is merely provided to aid the reader in understanding the disclosure and is not admitted to describe or constitute prior art thereto.

Water-absorbing gels, or hydrogels, are widely used in the biomedical field, for instance during viscosurgery and as a dermal filler. They are generally prepared by chemical crosslinking of polymers. Various injectable dermal filler products have been developed for treating or correcting facial imperfections, for example, wrinkles and volume loss due to the natural effects of aging. Injectable “dermal fillers” temporarily restore a smoother, more youthful appearance. Ideally, dermal fillers are long-lasting, soft, smooth and appear natural when introduced into or beneath the skin.

Glycosaminoglycans (GAGs) or mucopolysaccharides are long linear polysaccharides consisting of repeating disaccharide units (i.e. two-sugar units). The repeating two-sugar unit consists of a uronic sugar and an amino sugar. Hyaluronic acid (HA), also known as hyaluronan, is a water soluble glycosaminoglycan, which is a major component of the extra-cellular matrix and is widely distributed in animal tissues. HA has excellent biocompatibility and does not cause allergic reactions when implanted into a patient. In addition, HA has the ability to bind to large amounts of water, making it an excellent volumizer of soft tissues.

Alpha hydroxy acids (AHAs), are a class of chemical compounds that consist of a carboxylic acid substituted with a hydroxyl group on the adjacent carbon. Prominent examples are glycolic acid, lactic acid, and citric acid. Polymers made out of AHAs are useful as biocompatible materials. For instance, poly-L-lactic acid (PLLA) is an immunologically inert, biocompatible and biodegradable synthetic polymer. Once in the body, PLLA eventually degrades and undergoes resorption.

### SUMMARY OF THE INVENTION

The present application provides hydrogels and compositions that comprise a crosslinked glycosaminoglycan (e.g., hyaluronic acid or “HA”), an un-crosslinked glycosaminoglycan (e.g., HA), and a poly (alpha-hydroxy acid) polymer (e.g., poly-L-lactic acid or “PLLA”). The disclosed hydrogels possess improved flowability and spreadability compared to products that comprise, for example, crosslinking between HA and PLLA. The disclosed hydrogels and compositions can be used as dermal fillers or skin boosters and for treating or decreasing the appearance of fine lines, (e.g. fine lines on cheek) acne scars, and skin quality. The disclosed hydrogels boost the quality of the skin as a filler and stimulate the skin for immediate skin texture and quality with long lasting results, and the disclosed hydrogels and compositions are particularly well-suited for such uses due to its spreadability, which provides an immediate lifting effect for the skin, and sustained improvement of the skin quality and with long term implantation duration and stability/shelf-life. The hydrogels and compositions disclosed in the presentation application improves skin quality attributes including elasticity, radiance (reduced dry skin and increase hydration), texture and smoothness (improve pore size, crepiness) and skin tone for greater than or equal to 9 months duration (e.g., up to 12 or 18 months).

The disclosed hydrogels and compositions are particularly well-suited for such uses due to their spreadability, which provide an immediate lifting effect for the skin, and sustained improvement of the skin quality and with long term implantation duration and stability/shelf-life. The hydrogels and compositions disclosed in the presentation application improve skin quality attributes including improved elasticity, radiance (reduced dry skin and increase hydration), texture and smoothness (improve pore size, crepiness) and skin tone for between greater than or equal to 9 months duration, e.g., 9 months, 10 months, 11 months, 12 months, 13 months, 14 months, 15 months, 16 months, 17 months, 18 months, 19 months, 20 months, 21 months, 22 months, 23 months, or up to 24 months.

In one aspect, the present disclosure provides a composition comprising: (a) 10-45 mg/ml crosslinked glycosaminoglycan hydrogel, (b) 20%-50% free, non-crosslinked glycosaminoglycan by weight of the crosslinked glycosaminoglycan hydrogel, and (c) 10-50 mg/ml biodegradable poly (alpha-hydroxy acid) polymer, wherein the biodegradable poly (alpha-hydroxy acid) polymer is not crosslinked to the glycosaminoglycan hydrogel or the free, non-crosslinked glycosaminoglycan.

Another aspect of the disclosure provides a composition prepared by a process, comprising: (a) crosslinking a glycosaminoglycan in the presence of 2%-4% v/v NaOH, thereby obtaining a crosslinked glycosaminoglycan hydrogel; (b) filtering the crosslinked glycosaminoglycan hydrogel using a 60-90 micron filter; (c) adding to the filtered crosslinked glycosaminoglycan hydrogel 20%-50% free, non-crosslinked glycosaminoglycan by weight of the composition; and (d) adding to the composition from (c) 10-50 mg/ml biodegradable poly (alpha-hydroxy acid) polymer under vacuum conditions, wherein the biodegradable poly (alpha-hydroxy acid) polymer is not crosslinked to the glycosaminoglycan hydrogel. In some embodiments, the process further comprises swelling the crosslinked glycosaminoglycan hydrogel under vacuum conditions between (a) and (b). In some embodiments, the process further comprising precipitating and washing the filtered crosslinked glycosaminoglycan hydrogel from (b) using ethanol. In some embodiments, the process further comprises sterilizing the composition after (d).

In some embodiments, the composition comprises 10-20 mg/ml, 10-25 mg/ml, 10-30 mg/ml, 10-35 mg/ml, 10-40 mg/ml, 10-45 mg/ml, 15-20 mg/ml, 15-25 mg/ml, 15-30 mg/ml, 15-35 mg/ml, 15-40 mg/ml, or 15-45 mg/ml crosslinked glycosaminoglycan hydrogel.

In some embodiments, the composition comprises 20%-30%, 20%-35%, 20%-40% or 20%-45% free, non-crosslinked glycosaminoglycan by weight of the crosslinked glycosaminoglycan hydrogel.

In some embodiments, the composition comprises 10-15 mg/ml, 10-20 mg/ml, 10-25 mg/ml, 10-30 mg/ml, 10-35 mg/ml, 10-40 mg/ml or 10-50 mg/ml biodegradable poly (alpha-hydroxy acid) polymer.

In some embodiments, the crosslinked glycosaminoglycan hydrogel comprises crosslinked hyaluronic acid (HA), crosslinked heparan sulfate (HS), crosslinked heparin (HEP), crosslinked chondroitin sulfate (CS), crosslinked dermatan sulfate (DS), or crosslinked keratan sulfate (KS), or a combination thereof.

In some embodiments, the free glycosaminoglycan comprises hyaluronic acid (HA), heparan sulfate (HS), heparin (HEP), chondroitin sulfate (CS), dermatan sulfate (DS), or keratan sulfate (KS), or a combination thereof.

In some embodiments, the biodegradable poly (alpha-hydroxy acid) polymer comprises poly-L-lactic acid (PLLA), poly-D-lactic acid (PLDA), polyglycolic acid (PLGA) or poly (diol) citrate (PDC) or a combination thereof.

In some embodiments, the crosslinked glycosaminoglycan hydrogel has a size of about 60-90 microns and optionally wherein the biodegradable poly (alpha-hydroxy acid) polymer is about 40-63 microns.

In some embodiments, the crosslinked glycosaminoglycan is crosslinked by one or more polyfunctional crosslinking agent(s).

In some embodiments, the one or more polyfunctional crosslinking agent(s) are selected from the group consisting of divinyl sulfone, multiepoxides and diepoxides.

In some embodiments, the one or more polyfunctional crosslinking agent(s) are selected from the group consisting of 1,4-butanediol diglycidyl ether (BDDE), 1,2-ethanediol diglycidyl ether (EDDE) and diepoxyoctane.

In some embodiments, the crosslinked glycosaminoglycan is crosslinked by (i) a non-carbohydrate-based di- or multinucleofile crosslinker or (ii) a carbohydrate based di- or multinucleofile crosslinker.

In some embodiments, the non-carbohydrate-based di- or multinucleofile crosslinker is hexamethylenediamine (HMDA).

In some embodiments, the carbohydrate based di- or multinucleofile crosslinker is diaminotrehalose (DATH).

In some embodiments, the composition is an injectable composition.

Another aspect of the disclosure is directed to a method for improving skin quality in a subject in need of such treatment comprising administering the subject the composition of the instant disclosure.

Another aspect of the disclosure is directed to a method for preparing a composition, comprising: (a) crosslinking a glycosaminoglycan in the presence of 2%-4% v/v NaOH, thereby obtaining a crosslinked glycosaminoglycan hydrogel; (b) filtering the crosslinked glycosaminoglycan hydrogel using a 60-90 micron filter; (c) adding to the filtered crosslinked glycosaminoglycan hydrogel 20%-50% free glycosaminoglycan by weight of the composition; and (d) adding to the composition in step (c) 10-50 mg/ml biodegradable poly (alpha-hydroxy acid) polymer under vacuum conditions, wherein the biodegradable poly (alpha-hydroxy acid) polymer is not crosslinked to the glycosaminoglycan hydrogel.

In some embodiments, the method further comprises swelling the crosslinked glycosaminoglycan hydrogel under vacuum conditions between (a) and (b).

In some embodiments, the method further comprises precipitating and washing the filtered crosslinked glycosaminoglycan hydrogel from (b) using ethanol.

In some embodiments, the method further comprises sterilizing the composition after (d).

In some embodiments, the composition comprises 10-20 mg/ml, 10-25 mg/ml, 10-30 mg/ml, 10-35 mg/ml, 10-40 mg/ml, 10-45 mg/ml, 15-20 mg/ml, 15-25 mg/ml, 15-30 mg/ml, 15-35 mg/ml, 15-40 mg/ml, or 15-45 mg/ml crosslinked glycosaminoglycan hydrogel.

In some embodiments, the composition comprises 20%-30%, 20%-35%, 20%-40% or 20%-45% free glycosaminoglycan by weight of the crosslinked glycosaminoglycan hydrogel.

In some embodiments, the composition comprises 10-15 mg/ml, 10-15 mg/ml, 10-20 mg/ml, 10-25 mg/ml, 10-30 mg/ml, 10-35 mg/ml, 10-40 mg/ml or 10-45 mg/ml biodegradable poly (alpha-hydroxy acid) polymer.

In some embodiments, the crosslinked glycosaminoglycan hydrogel comprises crosslinked hyaluronic acid (HA), crosslinked heparan sulfate (HS), crosslinked heparin (HEP), crosslinked chondroitin sulfate (CS), crosslinked dermatan sulfate (DS), or crosslinked keratan sulfate (KS), or a combination thereof.

In some embodiments, the free glycosaminoglycan comprises hyaluronic acid (HA), heparan sulfate (HS), heparin (HEP), chondroitin sulfate (CS), dermatan sulfate (DS), or keratan sulfate (KS), or a combination thereof.

In some embodiments, the biodegradable poly (alpha-hydroxy acid) polymer comprises poly-L-lactic acid (PLLA), poly-D-lactic acid (PLDA), polyglycolic acid (PLGA) or poly (diol) citrate (PDC) or a combination thereof.

In some embodiments, the crosslinked glycosaminoglycan hydrogel has a size of about 60-90 microns and optionally wherein the biodegradable poly (alpha-hydroxy acid) polymer is about 40-63 microns.

In some embodiments, the crosslinked glycosaminoglycan is crosslinked by one or more polyfunctional crosslinking agent(s).

In some embodiments, the one or more polyfucntional crosslinking agent(s) are selected from the group consisting of divinyl sulfone, multiepoxides and diepoxides.

In some embodiments, the one or more polyfucntional crosslinking agent(s) are selected from the group consisting of 1,4-butanediol diglycidyl ether (BDDE), 1,2-ethanediol diglycidyl ether (EDDE) and diepoxyoctane.

In some embodiments, the crosslinked glycosaminoglycan is crosslinked by (i) a non-carbohydrate-based di- or multinucleofile crosslinker or (ii) a carbohydrate based di- or multinucleofile crosslinker.

In some embodiments, the non-carbohydrate-based di- or multinucleofile crosslinker is hexamethylenediamine (HMDA).

In some embodiments, the carbohydrate based di- or multinucleofile crosslinker is diaminotrehalose (DATH).

In some embodiments, the method further comprises formulating the composition as an injectable composition.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

The FIGURE (the FIG.) shows a generic representation of an example manufacturing process of the disclosed hydrogels.

### DETAILED DESCRIPTION

The present disclosure provides hydrogels and compositions that comprise a crosslinked glycosaminoglycan (e.g., hyaluronic acid or “HA”), an un-crosslinked glycosaminoglycan (e.g., HA), and a poly (alpha-hydroxy acid) polymer (e.g., poly-L-lactic acid or “PLLA”). The disclosed hydrogels possess firmness, flowability, and spreadability that make them ideal or cosmetic and dermatological uses including, but not limited to, treating or decreasing the appearance of fine lines and wrinkles. Moreover, the disclosed hydrogels and associated compositions may be stable for more than 18 months, and this shelf-life provides additional commercial desirability to the disclosed compositions, which may be transported in ampules, vials, or pre-filled syringes.

In general, the hydrogels and compositions of the disclosure are designed to deliver a constant PLLA dose during administration (e.g., injection) which is not possible with other hydrogels. In other words, the PPLA particles are distributed homogenously throughout the ampule/syringe/vial from the beginning, middle and end of ampule/syringe/vial. Upon administration to a subject, the hydrogels and compositions of the disclosure improve skin quality attributes including improved elasticity, radiance (reduced dry skin and increase hydration), texture and smoothness (improve pore size, reduce crepey skin) and skin tone for greater than or equal to 9 months duration, e.g., 9 months, 10 months, 11 months, 12 months, 13 months, 14 months, 15 months, 16 months, 17 months, 18 months, 19 months, 20 months, 21 months, 22 months, 23 months, or up to 24 months.

The disclosed hydrogels are formed using a process that prevents aggregation of the poly (alpha-hydroxy acid) polymer (e.g., PLLA) by preparing crosslinked GAG particles (e.g., crosslinked HA) within a desired size range to provide a more uniformly dispersed hydrogel and mixing the components under vacuum conditions during particular steps. Further, the addition of an un-crosslinked GAG (e.g., “free HA”) prevents undesirable stickiness that facilitates manufacturing, transportation, and ultimate use of the hydrogels and hydrogel-containing products. Taken together, the disclosed processed can produce a unique and desirable hydrogel that is suitable for any number of cosmetic and dermatological indications.

### Definitions

As used herein, the singular forms “a,” “an,” and “the” designate both the singular and the plural, unless expressly stated to designate the singular only.

It is to be understood, although not always explicitly stated, that all numerical designations are preceded by the term “about.” The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 15%, 10%, 5%, 1%, or 0.1% of the particular term.

Also as used herein, “and/or” refers to and encompasses any and all possible combinations of one or more of the associated listed items, as well as the lack of combinations when interpreted in the alternative (“or”).

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject. Administration can include without limitation, administration by subcutaneous, intramuscular, subdermal, intradermal, or transdermal injection. Administration can be unilateral or bilateral, as needed for a given patient.

### Hydrogels and Compositions

An aspect of the disclosure is directed to a composition comprising: 10-45 mg/ml crosslinked glycosaminoglycan hydrogel, 20%-50% free glycosaminoglycan by weight of the crosslinked glycosaminoglycan hyrdogel, and 10-50 mg/ml biodegradable poly (alpha-hydroxy acid) polymer, wherein the biodegradable poly (alpha-hydroxy acid) polymer is not crosslinked to the glycosaminoglycan hydrogel (i.e., it is a free biodegradable poly (alpha-hydroxy acid) polymer). In some embodiments, the crosslinked glycosaminoglycan hydrogel is a crosslinked hyaluronic acid (HA) hydrogel. In some embodiments, the free glycosaminoglycan is free hyaluronic acid (HA). In some embodiments, the biodegradable poly (alpha-hydroxy acid) polymer is poly-L-lactic acid (PLLA). Thus, in some embodiments, a disclosed composition may comprise 10-45 mg/ml crosslinked HA hydrogel, 20%-50% free HA by weight of the crosslinked HA hydrogel, and 10-50 mg/ml PLLA, wherein the PLLA is not crosslinked to the HA hydrogel.

An aspect of the disclosure is directed to a composition prepared by a process comprising: (a) crosslinking a glycosaminoglycan in the presence of 2%-4% (e.g., 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or 4%) v/v sodium hydroxide (NaOH), thereby obtaining a crosslinked glycosaminoglycan hydrogel; (b) filtering the crosslinked glycosaminoglycan hydrogel using a 60-90 micron filter (e.g., 60, 70, 80, or 90 micron filter); (c) adding to the filtered crosslinked glycosaminoglycan hydrogel 20%-50% (e.g., 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%) free glycosaminoglycan by weight of the crosslinked glycosaminoglycan hydrogel; and (d) adding to the composition in step (c) 5-60 mg/ml (5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg/ml) biodegradable poly (alpha-hydroxy acid) polymer under vacuum conditions, wherein the biodegradable poly (alpha-hydroxy acid) polymer is not crosslinked to the glycosaminoglycan hydrogel. In some embodiments, the process further comprises swelling the crosslinked glycosaminoglycan hydrogel under vacuum conditions between step (a) and step (b). In some embodiments, the process further comprises precipitating and washing the filtered crosslinked glycosaminoglycan hydrogel from step (b) using ethanol. In some embodiments, the process further comprises sterilizing the composition after step (d). In some embodiments the glycosaminoglycan is HA. In some embodiments, the biodegradable poly (alpha-hydroxy acid) polymer is PLLA.

In some embodiments, the crosslinking in step (a) is achieved in the presence of 2.3%-3.1% (e.g., 2.3%, 2.4%, 2.5%, 2.6%, 2.7%, 2.8%, 2.9%, 3%, or 3.1%) v/v NaOH. In some embodiments, the crosslinking in step (a) is achieved in the presence of 2.3%-2.5% (e.g., 2.3%, 2.4%, or 2.5%) v/v NaOH. In some embodiments, the crosslinking in step (a) is achieved in the presence of at least 2.4% NaOH.

In some embodiments, the composition is used as a dermal filler. The disclosed composition as a dermal filler displays improved flowability and spreadability when administered under the skin of a patient as a filler. The disclosed composition, when used as a dermal filler, also displays a more immediate and desirable “lift” due to the gel's increased firmness. Further, the disclosed hydrogels have low extrusion force (e.g., less than 12 Newton (12N) of force, e.g., 11N, 10N, 9N, 8N, 7N, 6N, or less) and cause minimal clogging while using a fine (e.g., a 25-30 gauge, such as a 25 gauge or a 27 gauge) needle. In addition, the disclosed hydrogels are ready to use, i.e., they do not require reconstitution before administration. HA-based filler products can be tested for firmness (G′) and flexibility (xStrain) using rheometry. G′ value reflects the gel strength (firmness) of a product. xStrain is an index of gel flexibility (how much strain a gel can withstand and still be reversible). In some embodiments, the disclosed hydrogels display a G′ value between 20 Pa and 200 Pa (e.g., 20, 30, 40, 50, 60, 70, 80, 90, 100, 110, 120, 130, 140, 150, 160, 170, 180, 190 or 200 Pa). In some embodiments, the disclosed hydrogels display a G′ value between 30 Pa and 100 Pa (e.g., 30, 40, 50, 60, 70, 80, 90, or 100 Pa).). In some embodiments, the disclosed hydrogels display a G′ value between 40 Pa and 80 Pa (e.g., 40, 50, 60, 70, or 80 Pa). In some embodiments, the disclosed hydrogels display an xStrain value between 250% and 500% (e.g., 250%, 260%, 270%, 280%, 290%, 300%, 310%, 320%, 330%, 350%, 360%, 370%, 380%, 390%, 400%, 410%, 420%, 430%, 450%, 460%, 470%, 480%, 490%, or 500%). In some embodiments, the disclosed hydrogels display an xStrain value between 300% and 400% (e.g., 300%, 310%, 320%, 330%, 350%, 360%, 370%, 380%, 390%, or 400%). In some embodiments, the disclosed hydrogels display an xStrain value between 310% and 380% (e.g., 310%, 320%, 330%, 350%, 360%, 370%, or 380%).

As used herein, the term “vacuum conditions” refers to conditions with barometric pressure of less than or equal to 30 mmHg. In some embodiments, vacuum conditions are achieved by a vacuum homogenizer. In some embodiments, the vacuum conditions also comprise temperatures of 68-72° C. (e.g., 68, 69, 70, 71, or 72° C.).

In some embodiments, the crosslinked glycosaminoglycan and the free glycosaminoglycan are comprised of the same type of glycosaminoglycan. In some embodiments, the crosslinked glycosaminoglycan is comprised of a first type of glycosaminoglycan and the free glycosaminoglycan is comprised of a second type of glycosaminoglycan, wherein the first type of glycosaminoglycan and the second type of glycosaminoglycan are different. In some embodiments, the first and second glycosaminoglycans are HA.

In some embodiments, the glycosaminoglycan is gamma irradiated. In some embodiments, the glycosaminoglycan is not gamma irradiated. In some embodiments, the glycosaminoglycan is heat sterilized.

In some embodiments, the composition comprises 5-20 mg/ml (e.g., 5, 6, 7, 8, 9, 10, 11, 12, 13, 14, 15, 16, 17, 18, 19, or 20 mg/ml), 5-15 mg/ml (e.g., 5, 6, 7, 8, 9, 10, 11, 12, 13, 14, or 15 mg/ml), 10-20 mg/ml (e.g., 10, 11, 12, 13, 14, 15, 16, 17, 18, 19, or 20 mg/ml), 10-25 mg/ml (e.g., 10, 11, 12, 13, 14, 15, 16, 17, 18, 19, 20, 21, 22, 23, 24, or 25 mg/ml), 10-30 mg/ml (e.g., 10, 11, 12, 13, 14, 15, 16, 17, 18, 19, 20, 21, 22, 23, 24, 25, 26, 27, 28, 29, or 30 mg/ml), 10-35 mg/ml (e.g., 10, 11, 12, 13, 14, 15, 16, 17, 18, 19, 20, 21, 22, 23, 24, 25, 26, 27, 28, 29, 30, 31, 32, 33, 34, or 35 mg/ml), 10-40 mg/ml (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/ml), 15-17 mg/ml (e.g., 15, 16, or 17 mg/ml), 15-20 mg/ml (e.g., 15, 16, 17, 18, 19, or 20 mg/ml), 15-25 mg/ml (e.g., 15, 16, 17, 18, 19, 20, 21, 22, 23, 24, or 25 mg/ml), 15-30 mg/ml (e.g., 15, 16, 17, 18, 19, 20, 21, 22, 23, 24, 25, 26, 27, 28, 29, or 30 mg/ml), 15-35 mg/ml (e.g., 15, 16, 17, 18, 19, 20, 21, 22, 23, 24, 25, 26, 27, 28, 29, 30, 31, 32, 33, 34, or 35 mg/ml), or 15-40 mg/ml (e.g., 15, 16, 17, 18, 19, 20, 21, 22, 23, 24, 25, 26, 27, 28, 29, 30, 31, 32, 33, 34, 35, 36, 37, 38, 39, or 40 mg/ml) crosslinked glycosaminoglycan (e.g., HA) hydrogel.

In some embodiments, the glycosaminoglycan is crosslinked with one or more crosslinkers, such as a polyfunctional crosslinking agent. The one or more polyfunctional crosslinking agent(s) individually selected from the group consisting of divinyl sulfone, multiepoxides and diepoxides. In certain embodiments, the polyfunctional crosslinking agent(s) is individually selected from the group consisting of 1,4-butanediol diglycidyl ether (BDDE), 1,2-ethanediol diglycidyl ether (EDDE) and diepoxyoctane. In some embodiments, the crosslinker is 1,4-butanediol diglycidyl ether (BDDE). Thus, in some embodiments, the crosslink comprises an ether bond. In some embodiments, the concentration of BDDE for crosslinking is between 0.001-0.05 g (e.g., 0.001, 0.005, 0.01, 0.02, 0.03, 0.04 or 0.05 g) BDDE per gram of glycosaminoglycan.

Additionally or alternatively, a crosslinker can comprise or consist of (i) the spacer group and (ii) binding groups formed upon reaction of the functional groups of the crosslinker with the carboxylic acid groups on the GAG. The spacer group may, for example, comprise a hyaluronic acid tetrasaccharide, hyaluronic acid hexasaccharide, trehalose, lactose, maltose, sucrose, cellobiose or raffinose residue. Thus, crosslinking can be achieved using a non-carbohydrate based di- or multinucleofile crosslinker, for example hexamethylenediamine (HMDA), or a carbohydrate based di- or multinucleofile crosslinker, for example diaminotrehalose (DATH) together with a glycosaminoglycan.

In some embodiments, the composition comprises 20%-30% (20%, 21%, 22%, 23%, 24%, 25%, 26%, 27%, 28%, 29%, or 30%), 20%-35% (20%, 21%, 22%, 23%, 24%, 25%, 26%, 27%, 28%, 29%, 30%, 31%, 32%, 33%, 34%, or 35%), 20%-40% (20%, 21%, 22%, 23%, 24%, 25%, 26%, 27%, 28%, 29%, 30%, 31%, 32%, 33%, 34%, 35%, 36%, 37%, 38%, 39% or 40%) or 20%-45% (20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45%) free glycosaminoglycan by weight of the crosslinked glycosaminoglycan hydrogel.

In some embodiments, the composition comprises 10-15 mg/ml (10, 11, 12, 13, 14 or 15 mg/ml), 10-20 mg/ml (10, 11, 12, 13, 14, 15, 16, 17, 18, 19, or 20 mg/ml), 10-25 mg/ml (10, 11, 12, 13, 14, 15, 16, 17, 18, 19, 20, 21, 22, 23, 24, or 25 mg/ml), 10-30 mg/ml (10, 11, 12, 13, 14, 15, 16, 17, 18, 19, 20, 21, 22, 23, 24, 25, 26, 27, 28, 29 or 30 mg/ml), 10-35 mg/ml (10, 11, 12, 13, 14, 15, 16, 17, 18, 19, 20, 21, 22, 23, 24, 25, 26, 27, 28, 29, 30, 31, 32, 33, 34, or 35 mg/ml), 10-40 mg/ml (10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/ml) or 10-45 mg/ml (10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 mg/ml) biodegradable poly (alpha-hydroxy acid) polymer. In some embodiments, the biodegradable poly (alpha-hydroxy acid) polymer is not gamma irradiated.

In some embodiments, the crosslinked glycosaminoglycan hydrogel comprises crosslinked hyaluronic acid (HA), crosslinked heparan sulfate (HS), crosslinked heparin (HEP), crosslinked chondroitin sulfate (CS), crosslinked dermatan sulfate (DS), or crosslinked keratan sulfate (KS), or a combination thereof. In some embodiments, the crosslinked glycosaminoglycan hydrogel comprises crosslinked hyaluronic acid (HA).

In some embodiments, the free glycosaminoglycan comprises hyaluronic acid (HA), heparan sulfate (HS), heparin (HEP), chondroitin sulfate (CS), dermatan sulfate (DS), or keratan sulfate (KS), or a combination thereof. In some embodiments, the free glycosaminoglycan comprises hyaluronic acid (HA).

In some embodiments, the crosslinked glycosaminoglycan hydrogel comprises crosslinked hyaluronic acid hydrogel and the free glycosaminoglycan comprises free hyaluronic acid.

In some embodiments, the biodegradable poly (alpha-hydroxy acid) polymer comprises poly-L-lactic acid (PLLA), poly-D-lactic acid (PLDA), polyglycolic acid (PLGA) or poly (diol) citrate (PDC) or a combination thereof. In some embodiments, the biodegradable poly (alpha-hydroxy acid) polymer comprises poly-L-lactic acid (PLLA).

In a specific embodiment, the composition comprises a hyaluronic acid (HA) gel that is crosslinked with 1,4-butanediol diglycidyl ether (BDDE), free HA (i.e., not crosslinked; about 35%), a phosphate buffer, about 0.7% sodium chloride, and free poly-L-lactic acid (PLLA) (i.e., not crosslinked).

In some embodiments, the crosslinked glycosaminoglycan hydrogel has a size of about 60-90 (e.g., about 60, 65, 70, 75, 80, 85, or 90) microns. In some embodiments, the term “about” refers to ±10% of a given value.

In some embodiments, the composition further comprises between 1 mg/ml and 15 mg/ml (e.g., 1, 2, 3, 4, 5, 6, 7, 8, 9, 10, 11, 12, 13, 14, or 15 mg/ml or any value in between) of a local anesthetic selected from lidocaine, bupivacaine, articaine, etidocaine or carbocaine. In a specific embodiment, the composition comprises 1 mg/ml and 15 mg/ml of lidocaine.

In some embodiments, the composition further comprises between 0.1% and 1.5% (e.g., 0.1%, 0.2%, 0.3%, 0.4%, 0.5%, 0.6%, 0.7%, 0.8%, 0.9%, 1%, 1.1%, 1.2%, 1.3%, 1.4% or 1.5%) sodium chloride.

In some embodiments, the composition has an osmolarity between about 250 mOsm/kg and 350 mOsm/kg (e.g., about 250 mOsm/kg, 275 mOsm/kg, 300 mOsm/kg, 325 mOsm/kg or 350 mOsm/kg).

In some embodiments, the composition further comprises between 9 mM and 20 mM (e.g., 9, 10, 11, 12, 13, 14, 15, 16, 17, 18, 19, or 20 mM) phosphate buffer.

In some embodiments, the composition is a sterilized composition. In some embodiments, the composition is an injectable composition. In some embodiments, the composition is packaged as a pre-filled syringe. In some embodiments, the composition can be used to treat fine lines and wrinkles, among other indications.

The disclosed compositions can be used in a method for treating wrinkles or fine lines in the skin of a subject in need of such treatment firming the skin of a subject in need of such treatment, or decreasing the appearance of wrinkles or fine lines in the skin of a subject. Such methods may generally comprise administering to the subject a composition of the instant disclosure via an injection (e.g., subcutaneous, subdermal, intradermal, transdermal, intramuscular, etc.). The injection may utilize a fine needle, such as a 25-30 gauge (G) needle (e.g., 25, 26, 27, 28, 29, or 30 G) needle. The needle may be about 0.5 inches long or longer. In some embodiments, the injection may be administered by a cannula.

### Methods for Preparing a Hydrogel Composition

Another aspect of the disclosure is directed to a method for preparing the composition disclosed herein. In some embodiments, the process of making the composition requires higher concentrations of crosslinkers, higher concentrations of sodium chloride, and higher concentrations of phosphate buffer as compared to comparable filler compositions. In some embodiments, the process also includes an additional purification/precipitation step prior to the addition of the free glycosaminoglycan and free biodegradable poly (alpha-hydroxy acid) polymer. An exemplary flow diagram illustrating an embodiment of the production process is provided in the FIGURE.

Another aspect of the disclosure is directed to a method for preparing a composition comprising: (a) crosslinking a glycosaminoglycan in the presence of 2%-4% v/v (e.g., 2%, 2.1%, 2.2%, 2.3%, 2.4%, 2.5%, 2.6%, 2.7%, 2.8%2, 2.9%, 3%, 3.1%, 3.2%, 3.3%, 3.4%, 3.5%, 3.6%, 3.7%, 3.8%, 3.9%, or 4%) NaOH, thereby obtaining a crosslinked glycosaminoglycan hydrogel; (b) filtering the crosslinked glycosaminoglycan hydrogel using a 60-90 micron filter; (c) adding to the filtered crosslinked glycosaminoglycan hydrogel 20%-50% (e.g., 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%) free glycosaminoglycan by weight of the composition; and (d) adding to the composition in step (c) 5-60 mg/ml (5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg/ml) biodegradable poly (alpha-hydroxy acid) polymer under vacuum conditions, wherein the biodegradable poly (alpha-hydroxy acid) polymer is not crosslinked to the glycosaminoglycan hydrogel.

In some embodiments, the crosslinking in step (a) is achieved in the presence of 2.3%-3.1% (e.g., 2.3%, 2.4%, 2.5%, 2.6%, 2.7%, 2.8%, 2.9%, 3%, or 3.1%) v/v NaOH. In some embodiments, the crosslinking in step (a) is achieved in the presence of 2.3%-2.5% (e.g., 2.3%, 2.4%, or 2.5%) v/v NaOH. In some embodiments, the crosslinking in step (a) is achieved in the presence of at least 2.4% NaOH.

In some embodiments, the process further comprises swelling the crosslinked glycosaminoglycan hydrogel under vacuum conditions between step (a) and step (b).

In some embodiments, vacuum conditions are achieved by a vacuum homogenizer. In some embodiments, the vacuum conditions also comprise temperatures of 68-72° C. (e.g., 68, 69, 70, 71, or 72° C.).

In some embodiments, the crosslinked glycosaminoglycan and the free glycosaminoglycan are comprised of the same type of glycosaminoglycan. In some embodiments, the crosslinked glycosaminoglycan is comprised of a first type of glycosaminoglycan and the free glycosaminoglycan is comprised of a second type of glycosaminoglycan, wherein the first type of glycosaminoglycan and the second type of glycosaminoglycan are different.

In some embodiments, the glycosaminoglycan is gamma irradiated. In some embodiments, the glycosaminoglycan is not gamma irradiated.

In some embodiments, the process further comprises precipitating and washing the filtered crosslinked glycosaminoglycan hydrogel from step (b) with ethanol. In some embodiments, the washed crosslinked glycosaminoglycan hydrogel is dried and swelled (rehydrated) before step (c).

In some embodiments, the process further comprises sterilizing the composition after step (d). In some embodiments, the sterilizing is achieved by autoclaving the composition. In some embodiments, the sterilizing is achieved by UV treatment.

In some embodiments, the crosslinking in step (a) is performed at ambient temperature between 21° C. and 25° C. (e.g., 21° C., 22° C. 23° C., 24° C. or 25° C.).

In some embodiments, the crosslinking in step (a) is achieved in between 16 h and 30 h (e.g., 16, 17, 18, 19, 20, 21, 22, 23, 24, 25, 26, 27, 28, 29, or 30 hours).

In some embodiments, the composition comprises 15-17 mg/ml (e.g., 15, 16, or 17 mg/ml), 15-20 mg/ml (e.g., 15, 16, 17, 18, 19, or 20 mg/ml), 15-25 mg/ml (e.g., 15, 16, 17, 18, 19, 20, 21, 22, 23, 24, or 25 mg/ml), 15-30 mg/ml (e.g., 15, 16, 17, 18, 19, 20, 21, 22, 23, 24, 25, 26, 27, 28, 29, or 30 mg/ml), 15-35 mg/ml (e.g., 15, 16, 17, 18, 19, 20, 21, 22, 23, 24, 25, 26, 27, 28, 29, 30, 31, 32, 33, 34, or 35 mg/ml), or 15-40 mg/ml (e.g., 15, 16, 17, 18, 19, 20, 21, 22, 23, 24, 25, 26, 27, 28, 29, 30, 31, 32, 33, 34, 35, 36, 37, 38, 39, or 40 mg/ml) crosslinked glycosaminoglycan hydrogel.

In some embodiments, the glycosaminoglycan is crosslinked using a crosslinker. In some embodiments, the crosslinker is 1,4-butanediol diglycidyl ether (BDDE). In some embodiments, the concentration of BDDE for crosslinking is between 0.001-0.05 g (e.g., 0.001, 0.005, 0.01, 0.02, 0.03, 0.04 or 0.05 g) BDDE per gram of glycosaminoglycan.

In some embodiments, the composition comprises 20%-30% (20%, 21%, 22%, 23%, 24%, 25%, 26%, 27%, 28%, 29%, or 30%), 20%-35% (20%, 21%, 22%, 23%, 24%, 25%, 26%, 27%, 28%, 29%, 30%, 31%, 32%, 33%, 34%, or 35%), 20%-40% (20%, 21%, 22%, 23%, 24%, 25%, 26%, 27%, 28%, 29%, 30%, 31%, 32%, 33%, 34%, 35%, 36%, 37%, 38%, 39% or 40%) or 20%-45% (20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45%) free glycosaminoglycan by weight of the composition.

In some embodiments, the composition comprises 10-15 mg/ml (10, 11, 12, 13, 14 or 15 mg/ml), 10-20 mg/ml (10, 11, 12, 13, 14, 15, 16, 17, 18, 19, or 20 mg/ml), 10-25 mg/ml (10, 11, 12, 13, 14, 15, 16, 17, 18, 19, 20, 21, 22, 23, 24, or 25 mg/ml), 10-30 mg/ml (10, 11, 12, 13, 14, 15, 16, 17, 18, 19, 20, 21, 22, 23, 24, 25, 26, 27, 28, 29 or 30 mg/ml), 10-35 mg/ml (10, 11, 12, 13, 14, 15, 16, 17, 18, 19, 20, 21, 22, 23, 24, 25, 26, 27, 28, 29, 30, 31, 32, 33, 34, or 35 mg/ml), 10-40 mg/ml (10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/ml) or 10-45 mg/ml (10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 mg/ml) biodegradable poly (alpha-hydroxy acid) polymer. In some embodiments, the biodegradable poly (alpha-hydroxy acid) polymer is not gamma irradiated.

In some embodiments, the crosslinked glycosaminoglycan and the free glycosaminoglycan are comprised of the same type of glycosaminoglycan. In some embodiments, the crosslinked glycosaminoglycan is comprised of a first type of glycosaminoglycan and the free glycosaminoglycan is comprised of a second type of glycosaminoglycan, wherein the first type of glycosaminoglycan and the second type of glycosaminoglycan are different.

In some embodiments, the crosslinked glycosaminoglycan hydrogel comprises crosslinked hyaluronic acid (HA), crosslinked heparan sulfate (HS), crosslinked heparin (HEP), crosslinked chondroitin sulfate (CS), crosslinked dermatan sulfate (DS), or crosslinked keratan sulfate (KS), or a combination thereof.

In some embodiments, the free glycosaminoglycan comprises hyaluronic acid (HA), heparan sulfate (HS), heparin (iEP), chondroitin sulfate (CS), dermatan sulfate (DS), or keratan sulfate (KS), or a combination thereof.

In some embodiments, the biodegradable poly (alpha-hydroxy acid) polymer comprises poly-L-lactic acid (PLLA), poly-D-lactic acid (PLDA), polyglycolic acid (PLGA) or poly (diol) citrate (PDC) or a combination thereof.

In some embodiments, the crosslinked glycosaminoglycan hydrogel has a size of about 60-90 (e.g., about 60, 65, 70, 75, 80, 85, or 90) microns. As used herein, the term, about refers to +10% of a given value.

In some embodiments, the biodegradable poly (alpha-hydroxy acid) polymer has a size of about 30-80 (e.g., about 30, 35, 40, 45, 50, 55, 60, 65, 70, 75, or 80) microns. In some embodiments, the biodegradable poly (alpha-hydroxy acid) polymer has a size of about 40-63 (e.g., about 40, 45, 50, 55, 60, or 63) microns.

In some embodiments, the composition further comprises between 0.1% and 1.5% (e.g., 0.1%, 0.2%, 0.3%, 0.4%, 0.5%, 0.6%, 0.7%, 0.8%, 0.9%, 1%, 1.1%, 1.2%, 1.3%, 1.4% or 1.5%) sodium chloride.

In some embodiments, the composition has an osmolarity between about 250 mOsm/kg and 350 mOsm/kg (e.g., about 250 mOsm/kg, 275 mOsm/kg, 300 mOsm/kg, 325 mOsm/kg or 350 mOsm/kg).

In some embodiments, the composition further comprises between 9 mM and 20 mM (e.g., 9, 10, 11, 12, 13, 14, 15, 16, 17, 18, 19, or 20 mM) phosphate buffer.

(In some embodiments, the composition is a sterilized composition. In some embodiments, the composition is an injectable composition. In some embodiments, the composition is packaged as a pre-filled syringe. In some embodiments, the composition can be used to treat fine lines and wrinkles, among other indications.

### EXAMPLES

An exemplary list of some of the components of the disclosed composition is shown in the Table 1 below.

TABLE 1
Example Composition

| Raw | | |
|---|---|---|
| Material/ | | |
| Component | Concentration | Comments |
| HA | 15-45 mg/ml | Notably increasing the gel |
| (Sodium | where of 35% | concentration lead to challenges in |
| Hyaluronic | added extra | getting a homogenous gel during |
| acid) | free HA | the swelling process due to |
| | | the cohesive nature of the gel. |
| Phosphate | 12 mM | Higher buffering capacity is |
| buffers | | needed compared to existing |
| | | products, as poly (alpha-hydroxy |
| | | acid) polymers (such as |
| | | PLLA) can be cleaved into acids, |
| | | which can affect the pH. |
| NaCl | 0.7 % | To obtain an osmolality of |
| | | about 300 mOsm/kg. |
| Lidocaine | 0 mg/ml | Pain reliever |
| Poly (alpha- | 10-50 mg/ml | Poly (alpha-hydroxy acid) |
| hydroxy | | polymers content (e.g., PLLA) |
| acid) | | affects the shelf life of the |
| polymer | | product, as higher content |
| | | generates more acid (lactic acid |
| | | in the case of PLLA), which |
| | | can lower the pH of the product. |
| | | The poly (alpha-hydroxy acid) |
| | | polymer content also affects |
| | | the clogging frequency where |
| | | increased content leads to more |
| | | stopping in the syringe. |
| | | The PLLA used (non-gamma |
| | | irradiated) has an inherent |
| | | viscosity 3.2-4.3 dl/g. Melting |
| | | range 170-200° C. |
| | | PLLA that was gamma irradiated |
| | | has an inherent viscosity |
| | | 0.7-1.1 dl/g. Both non-gamma |
| | | irradiated and gamma-irradiated |
| | | PLLA had similar particle size distribution. |

Table 2 further details process steps that were used and can be adapted to prepare the disclosed compositions. These methods are non-limited and may be adjusted as needed to account for desired properties of the resulting composition.

TABLE 2
Example Methods for Preparing Compositions

| Process Step | Method/Approach |
|---|---|
| Stabilizing | Stabilization has been carried out with |
| | both cooled and ambient NaOH, |
| | and guideline values during stabilization |
| | have been 23° C. for 24 h. |
| Swelling | Since the gel is very cohesive, a vessel |
| | with good mixing ability needs to |
| | be used to obtain homogeneous pH. |
| | Swelling has been diluted on a large |
| | scale to approximately 16 mg HA/ml |
| | of gel to facilitate mixing. |
| PSR (Particle | PSR is carried out by means of a pump |
| Size Reduction) | through a filter housing mounted |
| | with an 80 μm mesh to a receiving tank |
| | or directly to a folding vessel. To |
| | increase the yield, 0.9% NaCl solution |
| | is added to the swelling tank just |
| | before the end PSR to rinse out the system. |
| Precipitation | Using compressed air, 99.5% EtOH |
| | is transferred via a distribution arm |
| | into a precipitate vessel containing PSR gel. |
| Washing, | Gel powder slurry is transferred to a |
| dewatering | washing column by opening the |
| and | bottom valve of the precipitation vessel. |
| drying | In the washing column, gel |
| | powder has been washed in two |
| | batches in 70% EtOH, and finally |
| | dewatered in two batches with 99.5% |
| | EtOH. Drying has been carried out |
| | in the washing column overnight with |
| | vacuum and 35° C. support heating |
| | in the jacket. Gel powder from about |
| | 18 liters of gel has been washed and |
| | dried at the same time. |
| Swelling of gel | Swelling gel powder in buffer |
| powder | and adding extra HA. |
| Air removal | The bulk has been vacuum-drawn |
| | before the addition of PLLA as air |
| | bubbles can cause PLLA particles |
| | to aggregate form PLLA spheres. |
| Mixing in the | Mixing in the PLLA has been done |
| PLLA | by charging PLLA to the surface of |
| | the gel and starting stirring and |
| | gently restarting vacuum. After |
| | completion of the venture, the mixing |
| | in has lasted for at least 1 h until the |
| | gel has been visually homogeneous. |
| Filling and | The product is refilled with 1 ml |
| autoclaving | glass syringe and autoclaved at 125° C. |
| | for 5.5 minutes. |

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or systems, which can, of course, vary. It is also to be understood that the terminology used herein is for the purpose of describing particular embodiments only, and is not intended to be limiting.

What is claimed is:

1. A composition comprising:
(a) 10-45 mg/ml crosslinked glycosaminoglycan hydrogel,
(b) 20%-50% free, non-crosslinked glycosaminoglycan by weight of the crosslinked glycosaminoglycan hydrogel, and
(c) 10-50 mg/ml biodegradable poly (alpha-hydroxy acid) polymer, wherein the biodegradable poly (alpha-hydroxy acid) polymer is not crosslinked to the glycosaminoglycan hydrogel or the free, non-crosslinked glycosaminoglycan.

2. The composition of claim 1, wherein the composition comprises 15-40 mg/ml crosslinked glycosaminoglycan hydrogel.

3. The composition of claim 1, wherein the composition comprises 20%-45% free, non-crosslinked glycosaminoglycan by weight of the crosslinked glycosaminoglycan hydrogel.

4. The composition of claim 1, wherein the composition comprises 10-50 mg/ml biodegradable poly (alpha-hydroxy acid) polymer.

5. The composition of claim 1, wherein the crosslinked glycosaminoglycan hydrogel comprises crosslinked hyaluronic acid (HA), crosslinked heparan sulfate (HS), crosslinked heparin (HEP), crosslinked chondroitin sulfate (CS), crosslinked dermatan sulfate (DS), or crosslinked keratan sulfate (KS), or a combination thereof.

6. The composition of claim 1, wherein the free glycosaminoglycan comprises hyaluronic acid (HA), heparan sulfate (HS), heparin (HEP), chondroitin sulfate (CS), dermatan sulfate (DS), or keratan sulfate (KS), or a combination thereof.

7. The composition of claim 1, wherein the biodegradable poly (alpha-hydroxy acid) polymer comprises poly-L-lactic acid (PLLA), poly-D-lactic acid (PLDA), polyglycolic acid (PLGA) or poly (diol) citrate (PDC) or a combination thereof.

8. The composition of claim 1, wherein the crosslinked glycosaminoglycan hydrogel has a size of about 60-90 microns and wherein the biodegradable poly (alpha-hydroxy acid) polymer is about 40-63 microns.

9. The composition of claim 1, wherein the crosslinked glycosaminoglycan is crosslinked by one or more polyfunctional crosslinking agent(s).

10. The composition of claim 9, wherein the one or more polyfunctional crosslinking agent(s) are selected from the group consisting of divinyl sulfone, multiepoxides and diepoxides.

11. The composition of claim 9, wherein the one or more polyfunctional crosslinking agent(s) are selected from the group consisting of 1,4-butanediol diglycidyl ether (BDDE), 1,2-ethanediol diglycidyl ether (EDDE) and diepoxyoctane.

12. The composition of claim 1, wherein the crosslinked glycosaminoglycan is crosslinked by (i) a non-carbohydrate-based di- or multinucleofile crosslinker or (ii) a carbohydrate based di- or multinucleofile crosslinker.

13. The composition of claim 12, wherein the non-carbohydrate-based di- or multinucleofile crosslinker is hexamethylenediamine (HMDA) or the carbohydrate based di- or multinucleofile crosslinker is diaminotrehalose (DATH).

14. The composition of claim 1, wherein the composition is an injectable composition.

15. A composition prepared by a process, comprising:
(a) crosslinking a glycosaminoglycan in the presence of 2%-4% v/v NaOH, thereby obtaining a crosslinked glycosaminoglycan hydrogel;
(b) filtering the crosslinked glycosaminoglycan hydrogel using a 60-90 micron filter;
(c) adding to the filtered crosslinked glycosaminoglycan hydrogel 20%-50% free, non-crosslinked glycosaminoglycan by weight of the crosslinked glycosaminoglycan hydrogel; and
(d) adding to the composition from (c) 10-50 mg/ml biodegradable poly (alpha-hydroxy acid) polymer under vacuum conditions, wherein the biodegradable poly (alpha-hydroxy acid) polymer is not crosslinked to the glycosaminoglycan hydrogel.

16. The composition of claim 15, the process further comprising swelling the crosslinked glycosaminoglycan hydrogel under vacuum conditions between (a) and (b).

17. The composition of claim 15, the process further comprising precipitating and washing the filtered crosslinked glycosaminoglycan hydrogel from (b) using ethanol.

18. The composition of claim 15 further comprising sterilizing the composition after (d).

19. A method for improving skin quality in a subject in need of such treatment comprising administering the subject the composition of claim 1.

20. A method for preparing a composition, comprising:
(a) crosslinking a glycosaminoglycan in the presence of 2%-4% v/v NaOH, thereby obtaining a crosslinked glycosaminoglycan hydrogel;
(b) filtering the crosslinked glycosaminoglycan hydrogel using a 60-90 micron filter;
(c) adding to the filtered crosslinked glycosaminoglycan hydrogel 20%-50% free glycosaminoglycan by weight of the composition; and
(d) adding to the composition in step (c) 10-50 mg/ml biodegradable poly (alpha-hydroxy acid) polymer under vacuum conditions, wherein the biodegradable poly (alpha-hydroxy acid) polymer is not crosslinked to the glycosaminoglycan hydrogel.
